(19) United States
(12) Patent Application Publication
(10) Pub. No.: US 2014/0257015 A1
(43) Pub. Date: Sep. 11, 2014

(54) MODALITY WORK LIST SYSTEM

(71) Applicant: MEDRAD, INC., Indianola, PA (US)

(72) Inventors: James A. AGAMAITE, Wexford, PA (US); Larry McCUTCHAN, Allison Park, PA (US)

(73) Assignee: MEDRAD, INC., Indianola, PA (US)

(21) Appl. No.: 13/789,664

(22) Filed: Mar. 7, 2013

(51) Int. Cl. A61N 5/10

(52) U.S. Cl. CPC A61N 5/1007
USPC 600/4

(57) ABSTRACT

Methods and systems for automatically and dynamically determining a dose of a radiopharmaceutical are disclosed. The dose may be determined based on, among other things, radiopharmaceutical information associated with at least one source of a radiopharmaceutical, patient information and schedule information. An estimated radioactivity level may be determined based on an initial radioactivity level, a delivery time, a radioactivity decay rate, and an anticipated arrival time. A volume of the radiopharmaceutical to inject into a patient to deliver a dose of radioactivity may be determined based on the estimated radioactivity level and patient dosing information. An infusion apparatus may operate to inject the patient with the volume of the radiopharmaceutical.

### BACKGROUND

Radiopharmaceuticals are radioactive drugs used for medical diagnosis and disease therapy. Each day in the United States, radiopharmaceuticals are used in nearly 60,000 nuclear medicine procedures. The radioactivity of a particular volume of a radiopharmaceutical decreases with time, as a consequence of radioactive decay. The radioactivity decay is the “half-life” of the pharmaceutical, which is generally the time required for the radioactivity to decrease to half its value. Radiopharmaceuticals typically have very short half-lives, some as brief as two hours.

Radiopharmaceuticals may be delivered to a nuclear medicine department of a healthcare facility in a ready-to-use form generated by an outside manufacturer, such as a central radiopharmacy, or in a facility radiopharmacy. Once the radiopharmaceutical is formulated, it starts to lose radioactivity. As such, the volume of the radiopharmaceutical required to deliver a specific dose of radioactivity to a patient changes with time.

A multitude of factors are associated with scheduling a nuclear medicine patient and preparing the radiopharmaceutical. However, conventional health care facilities do not adequately manage all of the information required to administer a correct dose of a radiopharmaceutical to a patient. This is especially true when procedure schedules do not go precisely as planned, which is common. Consequently, it is difficult for medical personnel to efficiently and dynamically determine the dose for a particular patient at a particular time of day.

### SUMMARY

The invention described in this document is not limited to the particular systems, methodologies or protocols described, as these may vary. The terminology used herein is for the purpose of describing particular embodiments only, and is not intended to limit the scope of the present disclosure.

It must be noted that as used herein and in the appended claims, the singular forms “a,” “an,” and “the” include plural reference unless the context clearly dictates otherwise. Unless defined otherwise, all technical and scientific terms used herein have the same meanings as commonly understood by one of ordinary skill in the art. As used herein, the term “comprising” means “including, but not limited to.”

In an embodiment, a system for determining a dose of a radiopharmaceutical comprises a processor and one or more non-transitory, computer-readable storage mediums. The one or more non-transitory, computer-readable storage mediums contain radiopharmaceutical information associated with at least one source of a radiopharmaceutical, patient information, and schedule information. The radiopharmaceutical information may comprise an initial radioactivity level, a radioactivity decay rate and a delivery time. The patient information may comprise a patient identifier and patient dosing information. The schedule information may comprise an anticipated arrival time. At least one of the one or more computer-readable storage mediums may be in operable communication with the processor and may contain one or more programming instructions that, when executed, cause the processor to: receive the radiopharmaceutical information, the patient information, and the schedule information, determine an estimated radioactivity level based on the initial radioactivity level, the delivery time, the radioactivity decay rate, and the anticipated arrival time, and determine a volume of the radiopharmaceutical to inject into a patient to deliver a dose of radioactivity based on the estimated radioactivity level and the patient dosing information.

In an embodiment, a method for determining a dose of a radiopharmaceutical may comprise receiving, for storage in one or more non-transitory, computer-readable storage mediums: radiopharmaceutical information associated with at least one volume of a radiopharmaceutical, patient information and schedule information. The radiopharmaceutical information may comprise an initial radioactivity level, a radioactivity decay rate and a delivery time. The patient information may comprise a patient identifier and patient dosing information. The schedule information may comprise an anticipated arrival time. The method may further comprise determining, by a processor in operable communication with the one or more non-transitory, computer-readable storage mediums, an estimated radioactivity level of a radiopharmaceutical based on the initial radioactivity level, the delivery time, the radioactivity decay rate, and the anticipated arrival time. The processor may further determine a volume of the radiopharmaceutical to inject into a patient to deliver a dose of radioactivity based on the estimated radioactivity level and the patient dosing information.

### BRIEF DESCRIPTION OF THE DRAWINGS

FIG. 1 depicts an illustrative radiopharmaceutical dose management system according to some embodiments.

FIG. 2 depicts illustrative healthcare information according to some embodiments.

FIG. 3 depicts a flow diagram of a method for determining a dose of a radiopharmaceutical according to an embodiment.

FIG. 4 depicts a block diagram of illustrative internal hardware that may be used to contain or implement program instructions according to an embodiment.

### DETAILED DESCRIPTION

The terminology used in the description is for the purpose of describing the particular versions or embodiments only, and is not intended to limit the scope.

The present disclosure is directed toward automatically and dynamically determining proper radiopharmaceutical doses for a nuclear medicine procedure based on multiple information sources. In an embodiment, a healthcare information system may store information associated with, among other things, a radiopharmaceutical, a schedule, a patient, a procedure, and a radioactivity dose. A processor may be configured to receive the healthcare information. According to some embodiments, the processor may execute one or more software applications, such as a radiopharmaceutical dose control application. The radiopharmaceutical dose control application may use the healthcare information to determine the volume of a radiopharmaceutical to inject into a patient to achieve a proper dose. The determination of the proper injection volume may be based on various factors, including, without limitation, the radioactive decay rate (e.g., half-life) of the radiopharmaceutical, a delivery time of the radiopharmaceutical, the time of the procedure, and the prescribed dose of radioactivity. In one embodiment, the radiopharmaceutical dose application may further determine, based on the injection volume, whether there is an adequate amount of the radiopharmaceutical available in a source container.

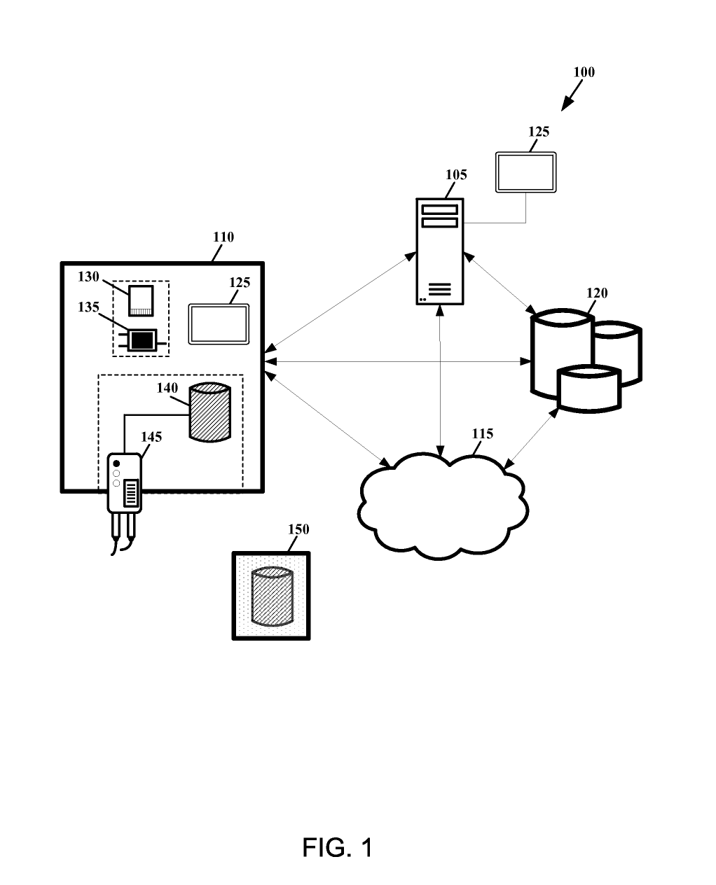

FIG. 1 depicts an illustrative radiopharmaceutical dose management system (“management system”) according to an embodiment. As shown in FIG. 1, a management system 100 may include an automated infusion apparatus 110 comprised of various components configured to deliver a dose of a radiopharmaceutical to a patient, such as a dispensing element 145 in fluid communication with a radiopharmaceutical source container 140. The dispensing element 145 may comprise any type of element capable of delivering the dose to the patient, such as intravenously through a syringe, catheter, needle, or automated injection system. The radiopharmaceutical source container 140 may be in the form of a shielded vial, commonly referred to as a “pig,” such as a lead or tungsten shielded vial. Illustrative and non-restrictive examples of radiopharmaceuticals include ⁶⁴Cu diacetyl-bis(N4-methylthiosemicarbazone) (ATSM or Copper 64), ¹⁸F-fluorodeoxyglucose (FDG), ¹⁸F-fluoride, 3′-deoxy-3′-[¹⁸F]fluorothymidine (FLT), ¹⁸F-fluoromisonidazole (FMISO), gallium, technetium-99m (^{99m}Tc), indium-113m (^{113m}In), strontium-87m (^{87m}Sr), and thallium. The radiopharmaceutical may be forced from the radiopharmaceutical source 140 to the dispensing element 145 and into the patient using methods known to those having ordinary skill in the art, such as being pumped using an infusion pump (not shown). Non-limiting examples of automated fluid delivery systems include the Intego™, Nautilus™ or Algernon™ infusion systems provided by Medrad, Inc. of Indianola, Pa.

In an embodiment, the radiopharmaceutical source 140 may comprise a self-contained volume of the radiopharmaceutical delivered as-is to a healthcare facility. For example, the radiopharmaceutical source 140 may comprise a syringe or a vial containing a volume of the radiopharmaceutical formulated or derived from a larger volume of the radiopharmaceutical formulated by a manufacturer or radiopharmacy. In another embodiment, the radiopharmaceutical source 140 may comprise a volume of the radiopharmaceutical obtained from a radiopharmaceutical bulk source 150 delivered to the healthcare facility. For instance, the radiopharmaceutical in the bulk source 150 may be transferred to the radiopharmaceutical source 140 as necessary using methods known to those having ordinary skill in the art.

One or more sensors (not shown) may be provided within the management system 100 to measure various properties associated with the radiopharmaceutical 140, 150 and/or operation of the infusion apparatus 110. The sensors may comprise any type of sensor capable of measuring a property of interest, including, without limitation, concentration, radioactivity, salinity, conductance, optical properties, analyte concentration, flow, and combinations thereof. Illustrative sensors include, but are not limited to, temperature sensors, pressure sensors, radioactivity sensors, optical sensors, analyte sensors, concentration sensors, flow sensors, electro-resistive devices, electro-capacitive devices, ultrasound devices, and combinations thereof. In an embodiment, radioactivity sensors may be provided for measuring the radioactivity of the radiopharmaceutical source container 140 and/or the radiopharmaceutical bulk source 150. In another embodiment, sensors may be provided for measuring the volume of the radiopharmaceutical source container 140 and/or the radiopharmaceutical bulk source 150.

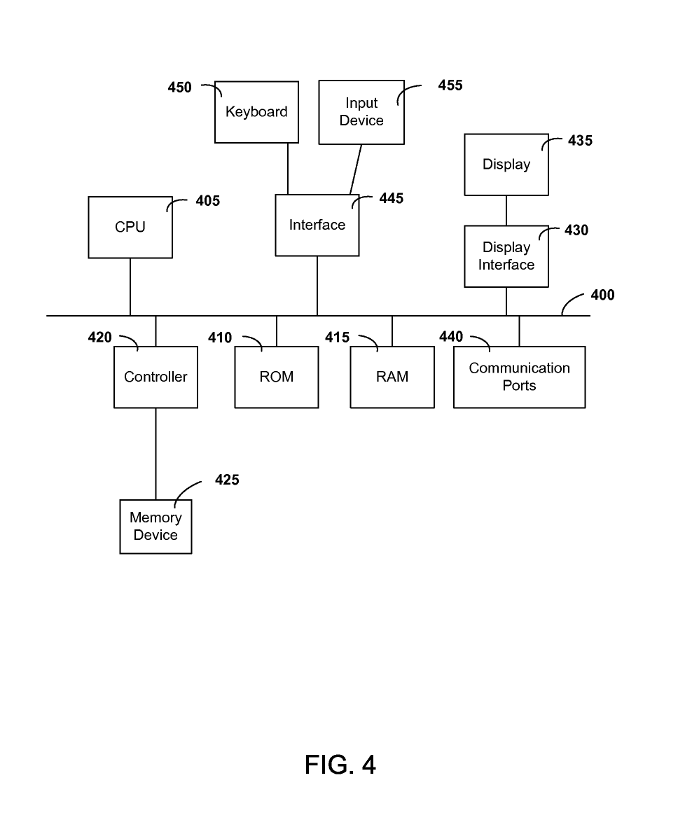

The infusion apparatus 110 may generally comprise one or more processors 135 and a non-transitory memory 130 or other storage device for storing programming instructions, one or more software programs, data or information regarding one or more applications, and other hardware, which may be the same or similar to the central processing unit (CPU) 405, read only memory (ROM) 410, random access memory 415, communication ports 440, controller 420, and/or memory device 425 depicted in FIG. 4 and described below in reference thereto.

The one or more processors 135 may be in communication with various elements of the infusion apparatus 110, including, without limitation, the dispensing element 125 and any sensors arranged within the infusion apparatus. The one or more processors 135 may execute one or more software programs, such as a radiopharmaceutical dose control application (“control application”), configured to determine, among other things, a radiopharmaceutical dose and/or injection volume for a patient based on information available within the management system 100. The control application may operate to determine a radioactivity concentration of a radiopharmaceutical (e.g., radioactivity/volume) based on a known radioactivity (e.g., based on user input and/or radioactivity measured by a radioactivity sensor) and the volume of the source radiopharmaceutical 140, 150. In a further embodiment, the control application may determine whether there is an adequate volume of a radiopharmaceutical to perform a procedure.

According to some embodiments, the control application may be configured to present a radiopharmaceutical dose user interface (“user interface”) on a display device 125. In one embodiment, the user interface may present information associated with a dose and/or volume of a radiopharmaceutical scheduled for delivery to a patient. In another embodiment, the user interface may present functions to specify the dose and/or volume of a radiopharmaceutical.

The management system 100 may comprise one or more computing devices 105. The computing devices 105 may comprise a processor and a non-transitory memory the same or substantially similar to the central processing unit (CPU) 405, read only memory (ROM) 410, random access memory 415, communication ports 440, controller 420, and/or memory device 425 depicted in FIG. 4 and described below in reference thereto. The computing device 105 may comprise various types of computing devices, including, without limitation, a server, personal computer (PC), tablet computer, computing appliance, or smart phone device.

The computing devices 105 may operate to execute the control application. In an embodiment, the computing device 105 and the infusion apparatus 110 may execute the same or substantially the same version (e.g., level, release, revision, module, etc.) of the control application. In another embodiment, the computing device 105 may execute a server version and the infusion apparatus may execute a client version. For example, the server version may be a more robust version of the control application configured to receive and transmit data, perform calculations, control access, and/or manage client applications. The client version, for instance, may be configured to receive information from the server application for display on the user interface and to receive local user input and/or information for transmission to the server application. Non-limiting examples of local information include radioactivity and volume information associated with a radiopharmaceutical source 140.

The management system 100 may include a network 115 configured to connect various types of electronic devices, computing devices 105 and information sources. The network 115 devices may include various types of computing devices 105, including, without limitation, a server, personal computer (PC), tablet computer, computing appliance, or smart phone device. Non-restrictive examples of networks 115 include, without limitation, communications networks or health information networks (e.g., picture archiving and communications system (PACS), health information systems (HIS), radiology information systems (RIS), or the like).

The management system 100 may include health information 120 comprised of various information associated with a healthcare facility or other entity managing nuclear medicine procedures. According to some embodiments, the health information 120 may be stored in databases, application data, electronic files, and/or other elements configured to store information known to those having ordinary skill in the art. The health information 120 may comprise various sources of information, including, without limitation, information associated with patient scheduling, patient information (e.g., name, age, weight, medical history, etc.), medical diagnostic and/or therapeutic procedures, radiopharmaceuticals, medical equipment, a healthcare facility, and/or scheduling rules. The health information 120 may be arranged in various collections and/or electronic storage containers (e.g., files, databases, etc.), including, but not limited to, a patient schedule container, a dose schedule container, and a medical equipment container, as described in more detail below. According to some embodiments, all or part of the health information 120 may be stored at various locations within the management system 100, including, without limitation, the non-transitory memory 130, computing devices 105, the network 115, and a health information management system (e.g., PACS) (not shown).

As shown in FIG. 1, all or some of the components of the management system 100 may be communicatively coupled to each other or capable of receiving/transmitting information from/to each other (e.g., through a shared or central network or other communication system connection). For example, the infusion apparatus 110 may comprise one or more communication ports (not shown) that provide communication with the computing devices 105, networks 115 and/or health information 120 storage locations. The communications ports may provide a connection through communication protocols known to those having ordinary skill in the art, such as serial, Ethernet and Wi-Fi. In another example, the computing devices 105 may be communicatively coupled to the network 115 and/or the health information 120 storage locations. In this manner, information, including the health information 120, may be accessed at various locations within the management system 100.

Hospitals and other healthcare facilities that carry out nuclear medicine imaging and therapeutic procedures may have restrictions on when they are able to receive the radiopharmaceutical agents for dispensing. As a result, they may operate to schedule a group of patients in one day to participate in the same procedure or different procedures that use the same radiopharmaceutical. Certain healthcare personnel, such as a physician, may determine the amount of the radiopharmaceutical to deliver to each patient based on certain factors. In an embodiment, the amount of the radiopharmaceutical may be measured in terms of total activity (e.g., total radioactivity). This total activity may be translated into a volume of the radiopharmaceutical, for example, drawn by an injector from a bulk supply having some initial activity concentration (e.g., disintegrations/sec/ml of fluid). Other healthcare personnel, such as a health care facility office manager, may be tasked with scheduling the patients. Because the radioactivity of the radiopharmaceutical decays over time, the required volume of a radiopharmaceutical delivered to a patient to achieve the required dose may be greater at a later time than a time earlier in the day. Accordingly, some embodiments provide for managing information associated with administering a radiopharmaceutical and for determining a dose of a radiopharmaceutical based on the information.

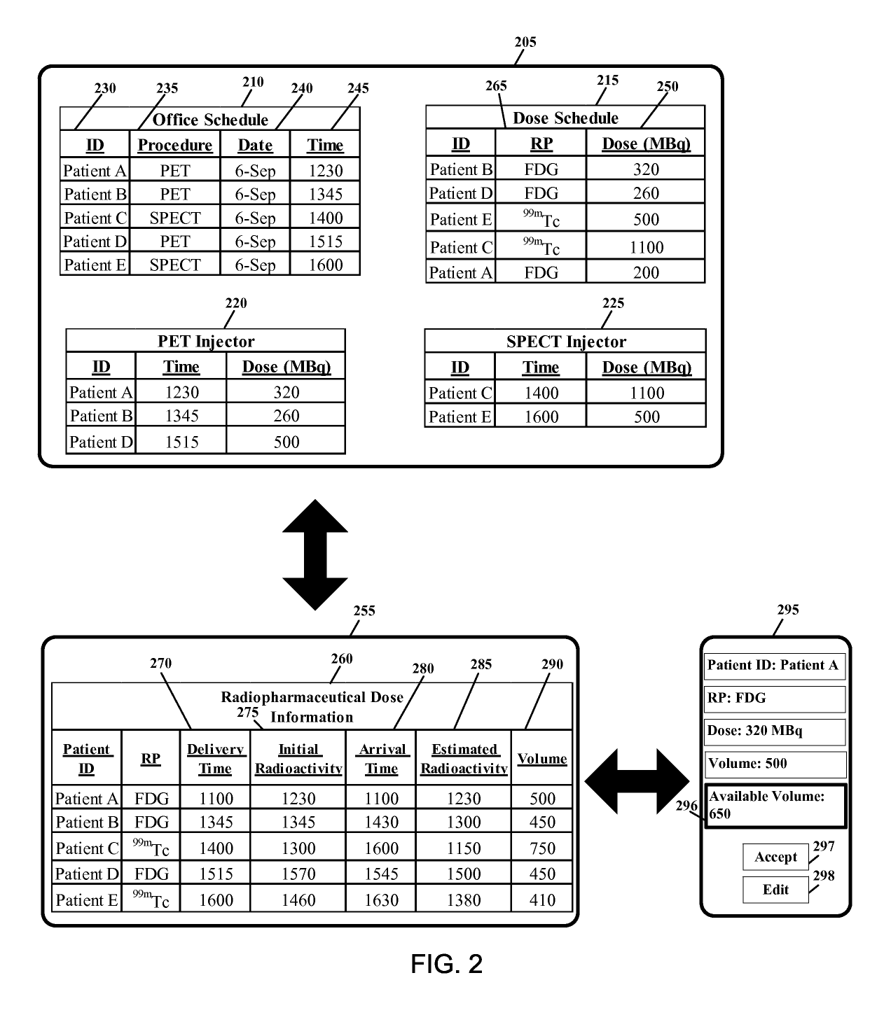

FIG. 2 depicts illustrative healthcare information according to some embodiments. As shown in FIG. 2, healthcare information may be categorized in various ways, such as into scheduling information 205 and radiopharmaceutical information 255. Within each of these categories, the healthcare information may be stored in various containers (e.g., electronic storage constructs, including files, databases, etc.), including an office schedule 210, a dose schedule 215, radiopharmaceutical dose information 260, and medical device schedules 220, 225. Illustrative medical device schedules include a positron emission tomography (PET) injector schedule 220 and a single-photon emission computed tomography (SPECT) injector schedule 225.

The categories and containers may be formed from any type of information storage element known to those having ordinary skill in the art that is capable of operating according to embodiments described herein, including, without limitation, databases, database tables, email system scheduling elements, spreadsheet application files, word processor application files, and scheduling software application files, such as BioRx by BioDose, LLC of Las Vegas, Nev. or Syntrac® Integration Tools by Cardinal Health of Dublin, Ohio.

Although the health information is depicted in FIG. 2 as being in certain categories and containers, embodiments are not so limited, as these are provided for illustrative purposes only. The health information may be arranged according to any configuration capable of operating according to embodiments described herein. In addition, embodiments may comprise more or less health information than depicted in FIG. 2. Furthermore, elements of the health information (e.g., records, values, calculations, etc.) may be duplicated across categories and/or containers.

The schedule information 205 may comprise various information pertaining to scheduling patients and/or resources within a healthcare facility. For example, the office schedule 210 may be developed as a schedule of nuclear medicine patients. The office schedule 210 may include information associated with a patient identifier (ID) 230, procedure and/or medical equipment 235, a date of the procedure 240, and a time of the procedure (e.g., an anticipated arrival time) 245. According to some embodiments, the office schedule 210 may be tied to various information systems, such as a hospital or healthcare facility record system. The office schedule 210 is not limited to the specific information depicted in FIG. 2, as this is for illustrative purposes only. According to some embodiments, the office schedule 210 may include more or less information than depicted in FIG. 2. Other information stored in the office schedule 210 may include patient name, date of birth, medical record numbers, address, phone number, consent information, patient doctors, radiology personnel performing the procedure, and/or physician or healthcare personnel notes.

A dose schedule 215 may include information associated with the radiopharmaceutical dose that will be delivered to a patient during a procedure. The dose schedule 215 may comprise information associated with a patient identifier 230, a radiopharmaceutical 265, and a dose 250. The radiopharmaceutical (RP) 265 information may provide the actual type of radiopharmaceutical that will be used during a procedure. The dose 250 information may provide the dose, or amount of radioactivity, of the radiopharmaceutical that will be administered to the patient. The dose 250 information may be included in various forms as appropriate for a particular radiopharmaceutical and/or procedure, such as in megabecquerels (MBq) and/or millicuries (mCi). The dose schedule 215 is not limited to the information depicted in FIG. 2. Embodiments provide for other types of information in the dose schedule 215, such as patient name and/or procedure type.

Schedules may also be provided for other entities associated with the healthcare facility, including medical equipment, rooms, physicians and other healthcare personnel, and procedures. In FIG. 2, schedules are depicted for a PET injector 220 and a SPECT injector 225. Information included in the schedules for the PET injector 220 and the SPECT injector 225 may comprise a patient identifier 230, a procedure time 245 and a dose 250.

The control application may access the scheduling information 205 to generate information associated with administering radiopharmaceutical doses to patients. For instance, the control application may operate to provide radiopharmaceutical doses under real-time conditions, such as doses calculated based on actual patient arrival times as opposed to anticipated patent arrival times. For instance, information from the office schedule 210 may be merged with information from the dose schedule 215 to generate new information, such as the volume of the radiopharmaceutical that should be administered to the patient to achieve a required dose and/or a specific infusion apparatus dosing list. Patient information may be used to correlate information between the various categories 205, 255 and containers 210, 215, 220, 225, 260. Non-limiting examples of such patient information include the patient identifier 230 and/or time of the procedure 245.

As shown in FIG. 2, the control application may operate to generate radiopharmaceutical information 205 comprising radiopharmaceutical dose information 260. The radiopharmaceutical dose information 260 may include information associated with a radiopharmaceutical 265, a delivery time 270 of the radiopharmaceutical, an initial radioactivity 275 of the radiopharmaceutical, a patient arrival time 280, estimated radioactivity 285 of the radiopharmaceutical, and a volume 290 of the radiopharmaceutical required to dispense the required radioactivity to the patient. The volume may be expressed in various units, such as milliliters (ml), cubic centimeters (cc), etc. According to some embodiments, the control application may control an infusion apparatus (e.g., infusion apparatus 110) to deliver a volume of the radiopharmaceutical specified in the volume 290 to a patient.

According to some embodiments, the patient arrival time 280 may comprise the actual arrival time (i.e., when the patient actually arrived at the healthcare facility and was ready for the procedure) and/or an estimated arrival time. As such, the control application may calculate the volume 290 as an anticipated volume based on the anticipated patient arrival time 280 and/or as an actual volume based on the actual patient arrival time. Some or all of these values may be included in the radiopharmaceutical dose information 260. In an embodiment, the control application may calculate an initial value for the volume 290 based on anticipated information (e.g., anticipated patient arrival time 280). In another embodiment, the control application may operate to dynamically update the volume 290 based on changes to the health information 120. For example, if the procedure time 245 in the office schedule 210 is updated, a corresponding change in the volume 290 may be calculated and propagated within the health information 120 sources, including apparatus use logs and patient medical history logs. The procedure time 245 may be modified for various reasons, including changes to reflect actual patient arrival time 280 (e.g., earlier and later arrival times) and rescheduling situations. In another example, if the dose 250 is modified in the scheduling information 205, the control application may receive the new value and re-calculate any values associated therewith, such as the volume 290.

The control application may determine the estimated radioactivity 285 of the radiopharmaceutical based on the initial radioactivity 275 of the radiopharmaceutical at the time of delivery 270, the half-life of the radiopharmaceutical, and the current time. The delivery time 270 may comprise other start times, such as the time of formulation, as long as the radioactivity at the time is known (or estimated, if an estimation is used). The time may be obtained from any time source capable of providing time information for determining the estimated radioactivity 285. Illustrative time sources include facility time source (e.g., a central computing system time), the time at the device executing the control application (e.g., infusion apparatus 110, computing device 105, etc.), or a national standard time source (National Institute of Standards and Technology (NIST) time sources).

Information associated with the radiopharmaceutical 260, such as the half-life or radioactivity information, may be obtained from various information sources, including default values provided by the manufacturer or external information sources. The control application may use the estimated radioactivity 285 to determine the volume 290 of the radiopharmaceutical 265 to deliver to the patient to achieve the required dose 250 of radioactivity.

In an embodiment, dose volume may be determined by knowing the activity concentration in a source container according to the following: dose volume (ml)=dose activity (mCi)/container concentration (mCi/ml). The container concentration may be the activity remaining in the container/volume remaining in the container, where the activity remaining may be adjusted for radioactive decay over time for a given radiopharmaceutical.

In an embodiment, the control application may use default information to populate some of the radiopharmaceutical dose information 260. The default information may comprise pre-programmed information, values calculated based on at least some pre-programmed values, and/or values calculated based on pre-programmed values and information associated with a particular procedure, radiopharmaceutical, and/or patient, such as patient weight, height, and/or age.

In an embodiment, the control application may operate to present a user interface 295 on a display device (e.g., display device 125). The user interface 295 may be configured to present information associated with administering a dose of a radiopharmaceutical to a patient, such as patient identifier 230, radiopharmaceutical 265, dose 250, and volume 295 information. The user interface 295 may be presented at various display devices 125 within the management system 100. For example, the user interface 295 may be presented on a display device at the infusion apparatus 110 where the patient is scheduled to receive the pharmaceutical. In another example, the user interface 295 may be presented on a display device 125 at a location remote from the infusion apparatus 110, such as a computing device 105 in a control room and/or physician's office.

The user interface 295 may present one or more functions, such as an accept 297 and an edit 298 function. The accept 297 function may be used to accept the presented information for infusing a patient with the radiopharmaceutical. In an embodiment, a user may be required to select the accept function 297 before starting an infusion procedure or releasing the information into the health information 120. For example, if a user selects the accept function 297, an associated infusion apparatus may operate to administer a dose of the radiopharmaceutical according to the accepted information.

The edit function 298 may be used to edit some or all of the information presented through the user interface 295. In an embodiment, a user may select the edit function 298 through an input device (not shown) (e.g., mouse, touchscreen, keyboard, etc.) which will operate to display a user interface screen for changing information displayed on the user interface 295. For example, the user may edit the patient identifier 230 to present information for a different patient. In this manner, the user may change the scheduling of a patient from the user interface 295. According to some embodiments, if the user modifies the schedule of a patient, effectively changing the procedure time 245, the control application may operate to automatically update the dosing information for the newly scheduled procedure. For example, the control application may re-calculate the required volume 290 based on the new procedure time 245. In another example, the dose 250 may be edited by the user. In this example, the control application may operate to re-calculate the volume of the radiopharmaceutical required for the procedure based on the updated dose 250.

As shown in FIG. 2, the user interface 295 may present information associated with the available volume 296 of the radiopharmaceutical. According to some embodiments, the volume information 296 may be actual volume information (e.g., measured by a sensor), calculated volume information (e.g., based on an initial delivery volume minus the volume used during procedures), or some combination thereof. The volume information 296 may include information from various sources, such as a bulk source (e.g., radiopharmaceutical bulk source 150) and/or a source contained within an infusion apparatus (e.g., radiopharmaceutical source 140).

In an embodiment, the volume information 296 may be configured to indicate whether there is an adequate supply of the radiopharmaceutical. The control application may operate to monitor the volume of available radiopharmaceutical and to generate a warning if there is and/or potentially will be an inadequate amount of the radiopharmaceutical. For example, an alarm or visual indication may be generated at the user interface 295 if there is not enough of the radiopharmaceutical for a scheduled procedure. An illustrative visual indication may include highlighting the volume information 296 element on the user interface 295. Illustrative alarms may include audible alarms and/or email alerts.

In an embodiment, the control application may determine the total volume of the radiopharmaceutical required for scheduled procedures before they have been performed and determine whether there is an adequate supply of the pharmaceutical. Accordingly, the control application may dynamically determine the remaining volume as each patient is scheduled. For example, if a schedule has been prepared and a new patient is added and/or a patient is moved to an appointment later in the day, such that more of the radiopharmaceutical will be required, medical personnel may be alerted if there will not be an adequate supply of the radiopharmaceutical. In this manner, the healthcare facility can reschedule patients, order more of the radiopharmaceutical, and/or take other preventative measures to ensure that the supply of the pharmaceutical is not exhausted, particularly during a procedure.

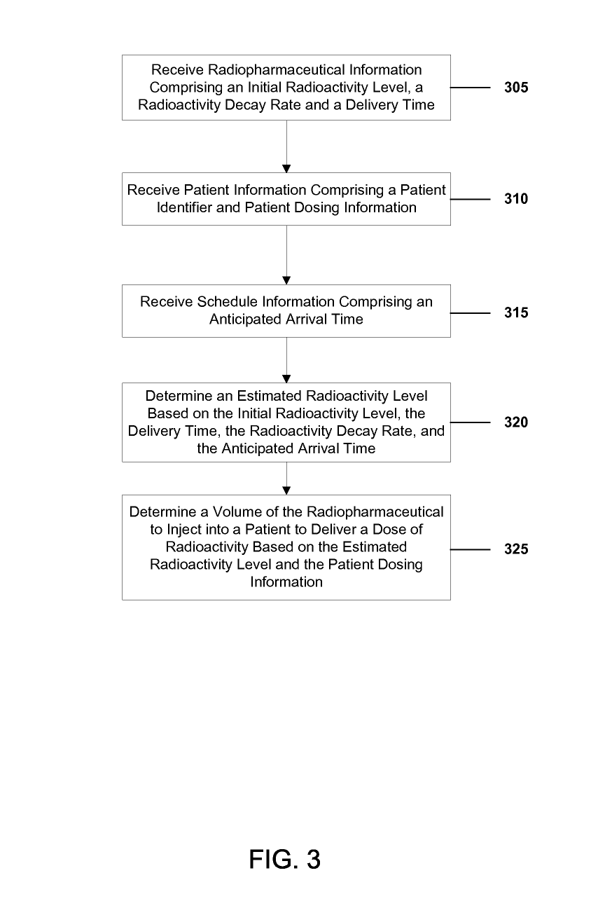

FIG. 3 depicts a flow diagram of a method for determining a dose of a pharmaceutical according to an embodiment. As shown in FIG. 3, radiopharmaceutical information may be received 305, for example, by a processor (e.g., processor 130 depicted in FIG. 1). The radiopharmaceutical information may comprise an initial radioactivity level, a radioactivity decay rate, and a delivery time. In one embodiment, the initial radioactivity level may include the radioactivity level of the radiopharmaceutical when it was and/or is scheduled to be delivered to the health care facility covered by the management system (e.g., management system 100). In another embodiment, the initial radioactivity level may comprise the radioactivity of the radiopharmaceutical when initially formulated. The radioactivity decay rate may comprise the half-life of the radiopharmaceutical as known to those having ordinary skill in the art. The delivery time may include the time that the radiopharmaceutical was delivered to the healthcare facility or the time that the radiopharmaceutical was formulated. The delivery time functions as a start time for determining the radioactivity of the radiopharmaceutical based on the radioactivity decay rate. As such, various delivery times (e.g., actual delivery, formulation, etc.) may be used, as long as the radioactivity at the delivery time (e.g., the initial radioactivity) is known.

Patient information may be received 310, at the processor, comprising a patient identifier and patient dosing information. The patient identifier may include any value capable of uniquely identifying a patient, including a social security number or a unique number assigned to each patient within the management system. The patient identifier may be used as a key to link all information associated with a patient to a unique patient record. The patient dosing information may comprise information associated with a dose of a radiopharmaceutical being administered to a patient. For example, the patient dosing information may include, without limitation, the type of radiopharmaceutical and the required radioactivity for the procedure.

Schedule information may be received 315, at the processor, comprising an anticipated arrival time. The schedule information may be associated with procedures scheduled at a healthcare facility within the management system. The anticipated arrival time may be configured to indicate the time that the patient is scheduled to undergo the procedure, such as a PET scan.

An estimated radioactivity level may be determined 320 based on the initial radioactivity level, the delivery time, the radioactivity decay rate, and the anticipated arrival time. The estimated radioactivity level may be configured to indicate the radioactivity of a source of the radiopharmaceutical (e.g., radiopharmaceutical source 140 and/or radiopharmaceutical bulk source 150) that will be used in a particular procedure. For example, the estimated radioactivity level for a known volume of radiopharmaceutical may be used to determine the volume of the pharmaceutical required to deliver a particular amount of radioactivity. The estimated radioactivity level may be determined by calculating how much the radioactivity of the radiopharmaceutical has decayed from the initial radioactivity level between the delivery time and the anticipated arrival time based on the radioactivity decay rate. In an embodiment, the following may be used to determine the estimated radioactivity level: A=A₀e^{-λt}. In this calculation, A is the current amount of radioactivity, A₀ is the original amount of radioactivity, e is the base natural log as known to those having ordinary skill in the art, λ is the decay constant 0.693/t_{1/2} (where t_{1/2} is the half-life of the radiopharmaceutical), and t is the time that has elapsed from A₀ to A.

In a non-limiting example in which the radiopharmaceutical is FPG, the decay rate may be configured as a half-life of about 109.77 minutes. The variable A may be the original activity of about 500 mCi assayed at about 12:00 p.m. (e.g., the delivery time). If the arrival time is about 2:00 p.m., the estimated radioactivity level may be given by the equation A₀e^{-λt}, where A₀=500 mCi, λ=0.693/109.77, t=120 minutes (e.g., the time difference between 12:00 p.m. and 2:00 p.m.), giving an activity estimate of about 234.4 mCi.

In an embodiment, the radioactivity level of a volume of a radiopharmaceutical may be measured using a radiation sensor as described herein. In such an embodiment, the measured radioactivity level may be used, among other things, to update and/or verify the initial radioactivity level and/or estimated radioactivity level.

The volume of the radiopharmaceutical to inject into a patient to deliver a dose of radioactivity may be determined 325 based on the estimated radioactivity level and the patient dosing information. For example, the estimated radioactivity level may be configured to indicate the volume of the radiopharmaceutical required to deliver a particular amount of radioactivity. The dose information may comprise the amount of radioactivity required for the procedure, such as 320 MBq. For example, the estimated radioactivity level may indicate that a particular volume of the radiopharmaceutical will deliver a particular amount of radiation if administered to a patient, such as X MBq/ml. The volume of the radiopharmaceutical to deliver to the patient may be determined by dividing the dose by the estimated radioactivity level as follows: (X MBq/mL)/Y MBq=Z ml required to deliver Y MBq.

FIG. 4 depicts a block diagram of exemplary internal hardware that may be used to contain or implement program instructions, such as the process steps discussed above in reference to FIG. 3, according to some embodiments. A bus 400 serves as the main information highway interconnecting the other illustrated components of the hardware. CPU 405 is the central processing unit of the system, performing calculations and logic operations required to execute a program. CPU 405, alone or in conjunction with one or more of the other elements disclosed in FIG. 1, is an exemplary processing device, computing device or processor as such terms are using in this disclosure. Read only memory (ROM) 410 and random access memory (RAM) 415 constitute exemplary memory devices.

A controller 420 interfaces with one or more optional memory devices 425 to the system bus 400. These memory devices 425 may include, for example, an external or internal DVD drive, a CD ROM drive, a hard drive, flash memory, a USB drive or the like. As indicated previously, these various drives and controllers are optional devices.

Program instructions, software or interactive modules for providing the digital marketplace and performing analysis on any received feedback may be stored in the ROM 410 and/or the RAM 415. Optionally, the program instructions may be stored on a tangible computer readable medium such as a compact disk, a digital disk, flash memory, a memory card, a USB drive, an optical disc storage medium, such as a Blu-ray™ disc, and/or other recording medium.

An optional display interface 430 may permit information from the bus 400 to be displayed on the display 435 in audio, visual, graphic or alphanumeric format. Communication with external devices may occur using various communication ports 440. An exemplary communication port 440 may be attached to a communications network, such as the Internet or an intranet. Other exemplary communication ports 440 may comprise a serial port, a RS-232 port, and a RS-485 port.

The hardware may also include an interface 445 which allows for receipt of data from input devices such as a keyboard 450 or other input device 455 such as a mouse, a joystick, a touch screen, a remote control, a pointing device, a video input device, and/or an audio input device.

It will be appreciated that various of the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which alternatives, variations and improvements are also intended to be encompassed by the following claims.

What is claimed is:

1. A system for determining a dose of a radiopharmaceutical, the system comprising:
a processor; and
one or more non-transitory, computer-readable storage mediums containing:
radiopharmaceutical information associated with at least one source of a radiopharmaceutical, the radiopharmaceutical information comprising an initial radioactivity level, a radioactivity decay rate and a delivery time,
patient information comprising a patient identifier and patient dosing information, and
schedule information comprising an anticipated arrival time,
wherein at least one of the one or more computer-readable storage mediums is in operable communication with the processor and contains one or more programming instructions that, when executed, cause the processor to:
determine an estimated radioactivity level based on the initial radioactivity level, the delivery time, the radioactivity decay rate, and the anticipated arrival time, and
determine a volume of the radiopharmaceutical to inject into a patient to deliver a dose of radioactivity based on the estimated radioactivity level and the patient dosing information.

2. The system of claim 1, wherein the radiopharmaceutical comprises one of the following: ⁶⁴Cu diacetyl-bis(N4-methylthiosemicarbazone), ¹⁸F-fluorodeoxyglucose (FDG), ¹⁸F-fluoride, 3′-deoxy-3′-[¹⁸F]fluorothymidine, ¹⁸F-fluoromisonidazole, gallium, technetium-99m (^{99m}Tc), indium-113m (^{113m}In), strontium-87m (^{87m}Sr), and thallium.

3. The system of claim 1, wherein the initial radioactivity level comprises a radioactivity of the source of the pharmaceutical at the delivery time.

4. The system of claim 1, wherein the delivery time comprises a time that the radiopharmaceutical was delivered.

5. The system of claim 1, wherein the delivery time comprises a time that the radiopharmaceutical was formulated.

6. The system of claim 1, wherein the radioactivity decay rate comprises a half-life of the radiopharmaceutical.

7. The system of claim 1, wherein the patient dosing information comprises a dose of radioactivity of the radiopharmaceutical.

8. The system of claim 1, wherein the radiopharmaceutical information further comprises a remaining volume of the at least one source of the radiopharmaceutical that is calculated by subtracting the volume from a current volume of the at least one source of the radiopharmaceutical.

9. A method for determining a dose of a radiopharmaceutical, the method comprising:
receiving, for storage in one or more non-transitory, computer-readable storage mediums:
radiopharmaceutical information associated with at least one volume of a radiopharmaceutical, the radiopharmaceutical information comprising an initial radioactivity level, a radioactivity decay rate and a delivery time,
patient information comprising a patient identifier and patient dosing information, and
schedule information comprising an anticipated arrival time,
determining, by a processor in operable communication with the one or more non-transitory, computer-readable storage mediums, an estimated radioactivity level of a radiopharmaceutical based on the initial radioactivity level, the delivery time, the radioactivity decay rate, and the anticipated arrival time; and
determining, by the processor, a volume of the radiopharmaceutical to inject into a patient to deliver a dose of radioactivity based on the estimated radioactivity level and the patient dosing information.

10. The method of claim 9, wherein the initial radioactivity level comprises a radioactivity of the source of the pharmaceutical at the delivery time.

11. The method of claim 9, wherein the delivery time comprises a time that the radiopharmaceutical was delivered.

12. The method of claim 9, wherein the delivery time comprises a time that the radiopharmaceutical was formulated.

13. The method of claim 9, wherein the radioactivity decay rate comprises a half-life of the radiopharmaceutical.

14. The method of claim 9, wherein the patient dosing information comprises a dose of radioactivity of the radiopharmaceutical.

15. The method of claim 9, further comprising calculating a remaining volume of the at least one source of the radiopharmaceutical by subtracting the volume from a current volume of the at least one source of the radiopharmaceutical.

16. The method of claim 15, further comprising monitoring the remaining volume.

17. The method of claim 15, further comprising updating the remaining volume responsive to determining the volume.

18. The method of claim 15, further comprising generating an alarm responsive to the volume being greater than the remaining volume.

19. The method of claim 9, further comprising presenting a radiopharmaceutical dose user interface on at least one display device in operable communication with the processor, the radiopharmaceutical dose user interface being configured to present the patient identifier, the patient dosing information and the volume.

20. The method of claim 19, further comprising configuring an infusion apparatus in operable communication with the processor to use the volume as an injection volume of the radiopharmaceutical to inject into the patient responsive to selection of the accept function.
